(19) United States
(12) Patent Application Publication
(10) Pub. No.: US 2002/0151541 A1
(43) Pub. Date: Oct. 17, 2002

(54) Pharmaceutical compositions containing tiotropium salts and antihistamines and their use

(76) Inventors: Michel Pairet, Stromberg (DE); Michael P. Pieper, Ingelheim (DE); Christopher John Montague Meade, Bingen (DE); Christel Schmelzer, Ingelheim (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/007,182

(22) Filed: Oct. 19, 2001

Related U.S. Application Data
(60) Provisional application No. 60/253,613, filed on Nov. 28, 2000.
(60) Provisional application No. 60/314,599, filed on Aug. 24, 2001.

(30) Foreign Application Priority Data
Oct. 31, 2000 (DE) 100 54 042.2
Aug. 10, 2001 (DE) 101 38 272.3

(51) Int. Cl. A61K 31/55; A61K 31/496; A61K 31/4743

(52) U.S. Cl. 514/217.05; 514/291; 514/649; 514/290; 514/255.04

(57) ABSTRACT

A pharmaceutical composition comprising a tiotropium salt; and an antihistamine, optionally together with a pharmaceutically acceptable excipient, the tiotropium salt and the antihistamine optionally in the form of their enantiomers, mixtures of their enantiomers, their racemates, their solvates, or their hydrates, processes for preparing them, and their use in the treatment of respiratory tract diseases.

### RELATED APPLICATIONS

Benefit under 35 U.S.C. §119(e) of prior provisional application Serial No. 60/253,613, filed Nov. 28, 2000, and prior provisional application Serial No. 60/314,599, filed Aug. 24, 2001, is hereby claimed.

### SUMMARY OF THE INVENTION

The present invention relates to novel pharmaceutical compositions based on tiotropium salts and antihistamines, processes for preparing them and their use in the treatment of respiratory diseases.

Surprisingly, it has been found that an unexpectedly beneficial therapeutic effect, particularly a synergistic effect can be observed in the treatment of diseases of the upper or lower respiratory tract, particularly in the treatment of allergic or non-allergic rhinitis, if one or more, preferably one anticholinergic is or are used together with one or more, preferably one, antihistamine.

The effects mentioned above are observed both when the two active substances are administered simultaneously in a single active substance formulation and when they are administered successively in separate formulations. According to the invention, it is preferable if the two active substance ingredients are administered simultaneously in a single formulation.

### DESCRIPTION OF THE DRAWINGS

FIG. 1 shows an exploded view of the Handihaler® inhaler for administering the pharmaceutical combination according to the invention in inhalettes;

FIG. 2a shows a longitudinal section of the Respimat® nebulizer disclosed in WO 97/12687 through the atomizer with the spring under tension; and

FIG. 2b shows a longitudinal section of the Respimat® nebulizer disclosed in WO 97/12687 through the atomizer with the spring released.

FIGS. 2a and 2b herein are identical to FIGS. 6a and 6b of WO 97/12687.

### DETAILED DESCRIPTION OF THE INVENTION

Within the scope of the present invention the term tiotropium salts 1 denotes salts which contain tiotropium, the pharmacologically active ingredient, as cation. Within the scope of the present patent application, any reference to the above cation is indicated by the use of the number 1′. Any reference to compounds 1 naturally also includes a reference to the ingredient 1′ (tiotropium).

By the salts 1 which may be used within the scope of the present invention are meant the compounds which contain, in addition to tiotropium as counter-ion (anion), chloride, bromide, iodide, methanesulfonate, p-toluenesulfonate or methylsulfate. Within the scope of the present invention, the methanesulfonate, chloride, bromide and iodide are preferred of all the salts 1, the methanesulfonate and bromide being of particular importance. Tiotropium bromide is of outstanding importance according to the invention as the salt 1.

Within the scope of the present invention the term antihistamines (hereinafter 2) denotes compounds selected from among epinastine, cetirizine, azelastine, fexofenadine, levocabastine, loratadine, mizolastine, ketotifen, emedastine, dimethindene, clemastine, bamipine, dexchlorpheniramine, pheniramine, doxylamine, chlorphenoxamine, dimenhydrinate, diphenhydramine, promethazine, ebastine, desloratadine and meclozine.

Preferably, compound 2 is selected from among epinastine, cetirizine, azelastine, fexofenadine, levocabastine, loratadine, ebastine, desloratadine and mizolastine, while epinastine and desloratadine are particularly preferred as compound 2 according to the invention. Most preferably, epinastine is used as compound 2 within the scope of the present invention. Any reference to the abovementioned antihistamines 2 within the scope of the present invention includes a reference to any pharmacologically acceptable acid addition salts thereof which may exist.

By the physiologically acceptable acid addition salts which may be formed from 2 are meant, according to the invention, pharmaceutically acceptable salts selected from among the salts of hydrochloric acid, hydrobromic acid, sulfuric acid, phosphoric acid, methanesulfonic acid, acetic acid, fumaric acid, succinic acid, lactic acid, citric acid, tartaric acid or maleic acid. Preferred salts of the compounds 2 are those selected from among the acetate, hydrochloride, hydrobromide, sulfate, phosphate and methanesulfonate. In this context, hydrochlorides and hydrobromides are particularly preferred. In the case of epinastine, which is particularly preferred according to the invention, epinastine hydrochloride is of exceptional importance.

The pharmaceutical combinations of 1 and 2 according to the invention are preferably administered by inhalation or by nasal application. Suitable inhalable powders packed into suitable capsules (inhalettes) may be administered using suitable powder inhalers. Alternatively, the drug may be inhaled by the application of suitable inhalation aerosols. These include inhalation aerosols which contain HFA134a (also known as TG134a), HFA227 (also known as TG227) or a mixture thereof as propellant gas. The drug may also be inhaled using suitable solutions of the pharmaceutical combination consisting of 1 and 2.

If the pharmaceutical combination of 1 and 2 is administered nasally, suitable solutions which may be administered by appropriate pumps can be used. Alternatively, it may be administered nasally by the application of suitable powders.

In one aspect, therefore, the invention relates to a pharmaceutical composition which contains a combination of 1 and 2.

In another aspect the present invention relates to a pharmaceutical composition which contains one or more salts 1 and one or more compounds 2, optionally in the form of their solvates or hydrates. The active substances may either be combined in a single preparation or contained in two separate formulations. Pharmaceutical compositions which contain the active substances 1 and 2 in a single preparation are preferred according to the invention. In another aspect the present invention relates to a pharmaceutical composition which contains, in addition to therapeutically effective quantities of 1 and 2, a pharmaceutically acceptable excipient. In another aspect the present invention relates to a pharmaceutical composition which does not contain any pharmaceutically acceptable excipient in addition to therapeutically effective quantities of 1 and 2.

The present invention also relates to the use of 1 and 2 for preparing a pharmaceutical composition containing therapeutically effective quantities of 1 and 2 for treating diseases of the upper or lower respiratory tract, particularly for treating allergic or non-allergic rhinitis.

The present invention further relates to the simultaneous or successive use of therapeutically effective doses of the combination of the above pharmaceutical compositions 1 and 2 for treating diseases of the upper or lower respiratory tract, particularly for treating allergic or non-allergic rhinitis.

In the active substance combinations of 1 and 2 according to the invention, ingredients 1 and 2 may be present in the form of their enantiomers, mixtures of enantiomers or in the form of racemates.

The proportions in which the two active substances 1 and 2 may be used in the active substance combinations according to the invention are variable. Active substances 1 and 2 may possibly be present in the form of their solvates or hydrates. Depending on the choice of the compounds 1 and 2, the weight ratios which may be used within the scope of the present invention vary on the basis of the different molecular weights of the various compounds and their different potencies. As a rule, the pharmaceutical combinations according to the invention may contain compounds 1 and 2 in ratios by weight ranging from 1:300 to 50:1, preferably from 1:250 to 40:1. In the particularly preferred pharmaceutical combinations which contain tiotropium salt as compound 1, the weight ratios of 1 to 2 are most preferably in a range in which tiotropium 1′ and 2 are present in proportions of 1:150 to 30:1, more preferably from 1:50 to 20:1.

For example, without restricting the scope of the invention thereto, preferred combinations of 1 and 2 according to the invention may contain tiotropium 1′ and antihistamine 2 in the following weight ratios: 1:80; 1:79; 1:78; 1:77; 1:76; 1:75; 1:74; 1:73; 1:72; 1:71; 1:70; 1:69; 1:68; 1:67; 1:66; 1:65; 1:64; 1:63; 1:62; 1:61; 1:60; 1:59; 1:58; 1:57; 1:56; 1:55; 1:54; 1:53; 1:52; 1:51; 1:50; 1:49; 1:48; 1:47; 1:46; 1:45; 1:44; 1:43; 1:42; 1:41; 1:40; 1:39; 1:38; 1:37; 1:36; 1:35; 1:34; 1:33; 1:32; 1:31; 1:30; 1:29; 1:28; 1:27; 1:26; 1:25; 1:24; 1:23; 1:22; 1:21; 1:20; 1:19; 1:18; 1:17; 1:16; 1:15; 1:14; 1:13; 1:12; 1:11; 1:10; 1:9; 1:8; 1:7; 1:6; 1:5; 1:4; 1:3; 1:2; 1:1; 2:1; 3:1; 4:1; 5:1; 6:1; 7:1; 8:1; 9:1; 10:1; 11:1; 12:1; 13:1; 14:1; 15:1; 16:1; 17:1; 18:1; 19:1; and 20:1.

The pharmaceutical compositions according to the invention containing the combinations of 1 and 2 are normally administered so that 1 and 2 are present together in doses of 0.01 μg to 10,000 μg, preferably from 0.1 μg to 2000 μg, more preferably from 1 μg to 1500 μg, better still from 50 μg to 1200 μg per single dose. For example, combinations of 1 and 2 according to the invention contain a quantity of tiotropium 1′ and antihistamine 2 such that the total dosage per single dose is 100 μg; 105 μg; 110 μg; 115 μg; 120 μg; 125 μg; 130 μg; 135 μg; 140 μg; 145 μg; 150 μg; 155 μg; 160 μg; 165 μg; 170 μg; 175 μg; 180 μg; 185 μg; 190 μg; 195 μg; 200 μg; 205 μg; 210 μg; 215 μg; 220 μg; 225 μg; 230 μg; 235 μg; 240 μg; 245 μg; 250 μg; 255 μg; 260 μg; 265 μg; 270 μg; 275 μg; 280 μg; 285 μg; 290 μg; 295 μg; 300 μg; 305 μg; 310 μg; 315 μg; 320 μg; 325 μg; 330 μg; 335 μg; 340 μg; 345 μg; 350 μg; 355 μg; 360 μg; 365 μg; 370 μg; 375 μg; 380 μg; 385 μg; 390 μg; 395 μg; 400 μg; 405 μg; 410 μg; 415 μg; 420 μg; 425 μg; 430 μg; 435 μg; 440 μg; 445 μg; 450 μg; 455 μg; 460 μg; 465 μg; 470 μg; 475 μg; 480 μg; 485 μg; 490 μg; 495 μg; 500 μg; 505 μg; 510 μg; 515 μg; 520 μg; 525 μg; 530 μg; 535 μg; 540 μg; 545 μg; 550 μg; 555 μg; 560 μg; 565 μg; 570 μg; 575 μg; 580 μg; 585 μg; 590 μg; 595 μg; 600 μg; 605 μg; 610 μg; 615 μg; 620 μg; 625 μg; 630 μg; 635 μg; 640 μg; 645 μg; 650 μg; 655 μg; 660 μg; 665 μg; 670 μg; 675 μg; 680 μg; 685 μg; 690 μg; 695 μg; 700 μg; 705 μg; 710 μg; 715 μg; 720 μg; 725 μg; 730 μg; 735 μg; 740 μg; 745 μg; 750 μg; 755 μg; 760 μg; 765 μg; 770 μg; 775 μg; 780 μg; 785 μg; 790 μg; 795 μg; 800 μg; 805 μg; 810 μg; 815 μg; 820 μg; 825 μg; 830 μg; 835 μg; 840 μg; 845 μg; 850 μg; 855 μg; 860 μg; 865 μg; 870 μg; 875 μg; 880 μg; 885 μg; 890 μg; 895 μg; 900 μg; 905 μg; 910 μg; 915 μg; 920 μg; 925 μg; 930 μg; 935 μg; 940 μg; 945 μg; 950 μg; 955 μg; 960 μg; 965 μg; 970 μg; 975 μg; 980 μg; 985 μg; 990 μg; 995 μg; 1000 μg; 1005 μg; 1010 μg; 1015 μg; 1020 μg; 1025 μg; 1030 μg; 1035 μg; 1040 μg; 1045 μg; 1050 μg; 1055 μg; 1060 μg; 1065 μg; 1070 μg; 1075 μg; 1080 μg; 1085 μg; 1090 μg; 1095 μg; 1100 μg, or the like. The proposed dosages per single dose suggested above are not to be regarded as being restricted to the numerical values actually stated, but are intended only as examples of dosages. Of course, dosages which fluctuate around the above values in a range of about ±2.5 μg are also covered by the values given above by way of example. In these dosage ranges the active substances 1′ and 2 may be present in the weight ratios specified above.

For example, without restricting the scope of the invention thereto, the combinations of 1 and 2 according to the invention may contain a quantity of tiotropium 1′ and antihistamine 2 such that, in each individual dose, 5 μg of 1′ and 25 μg of 2; 5 μg of 1′ and 50 μg of 2; 5 μg of 1′ and 100 μg of 2; 5 μg of 1′ and 200 μg of 2; 5 μg of 1′ and 300 μg of 2; 5 μg of 1′ and 400 μg of 2; 5 μg of 1′ and 500 μg of 2; 5 μg of 1′ and 600 μg of 2; 5 μg of 1′ and 700 μg of 2; 5 μg of 1′ and 800 μg of 2; 5 μg of 1′ and 900 μg of 2; 5 μg of 1′ and 1000 μg of 2; 10 μg of 1′ and 25 μg of 2; 10 μg of 1′ and 50 μg of 2; 10 μg of 1′ and 100 μg of 2; 10 μg of 1′ and 200 μg of 2; 10 μg of 1′ and 300 μg of 2; 10 μg of 1′ and 400 μg of 2; 10 μg of 1′ and 500 μg of 2; 10 μg of 1′ and 600 μg of 2; 10 μg of 1 and 700 μg of 2; 10 μg of 1′ and 800 μg of 2; 10 μg of 1′ and 900 μg of 2; 10 g of 1′ and 1000 μg of 2; 18 μg of 1′ and 25 μg of 2; 18 μg of 1′ and 50 μg of 2; 18 μg of 1′ and 100 μg of 2; 18 μg of 1′ and 200 μg of 2; 18 μg of 1′ and 300 μg of 2; 18 μg of 1′ and 400 μg of 2; 18 μg of 1′ and 500 μg of 2; 18 μg of 1′ and 600 μg of 2; 18 μg of 1′ and 700 μg of 2; 18 μg of 1′ and 800 μg of 2; 18 μg of 1′ and 900 μg of 2; 18 μg of 1′ and 1000 μg of 2; 20 μg of 1′ and 25 μg of 2; 20 μg of 1′ and 50 μg of 2; 20 μg of 1′ and 100 μg of 2; 20 μg of 1′ and 200 μg of 2; 20 μg of 1 and 300 μg of 2; 20 μg of 1 and 400 μg of 2; 20 μg of 1′ and 500 μg of 2; 20 μg of 1′ and 600 μg of 2; 20 μg of 1′ and 700 μg of 2; 20 μg of 1′ and 800 μg of 2; 20 μg of 1′ and 900 μg of 2; 20 μg of 1′ and 1000 μg of 2; 36 μg of 1′ and 25 μg of 2; 36 μg of 1′ and 50 μg of 2; 36 μg of 1′ and 100 μg of 2; 36 μg of 1′ and 200 μg of 2; 36 μg of 1′ and 300 μg of 2; 36 μg of 1′ and 400 μg of 2; 36 μg of 1′ and 500 μg of 2; 36 μg of 1′ and 600 μg of 2; 36 μg of 1′ and 700 μg of 2; 36 μg of 1′ and 800 μg of 2; 36 μg of 1′ and 900 μg of 2; 36 μg of 1′ and 1000 μg of 2; 40 μg of 1′ and 25 μg of 2; 40 μg of 1′ and 50 μg of 2; 40 μg of 1′ and 100 μg of 2; 40 μg of 1′ and 200 μg of 2; 40 μg of 1′ and 300 μg of 2; 40 μg of 1′ and 400 μg of 2; 40 μg of 1′ and 500 μg of 2 or 40 μg of 1′ and 600 μg of 2; 40 μg of 1′ and 700 μg of 2; 40 μg of 1′ and 800 μg of 2; 40 μg of 1′ and 900 μg of 2; 40 μg of 1′ and 1000 μg of 2 are administered.

If the active substance combination in which 1 denotes tiotropium bromide is used as the preferred combination of 1 and 2 according to the invention, the quantities of active substance 1′ and 2 administered per single dose mentioned by way of example correspond to the following quantities of 1 and 2 administered per single dose: 6 μg of 1 and 25 μg of 2; 6μg of 1′ and 50 μg of 2; 6 μg of 1 and 100 μg of 2; 6 μg of 1 and 200 μg of 2; 6 μg of 1 and 300 μg of 2; 6 μg of 1′ and 400 μg of 2; 6 μg of 1′ and 500 μg of 2; 6 μg of 1 and 600 μg of 2; 6 μg of 1 and 700 μg of 2; 6 μg of 1′ and 800 μg of 2; 6 μg of 1 and 900 μg of 2; 6 μg of 1 and 1000 μg of 2; 12 μg of 1 and 25 μg of 2; 12 μg of 1 and 50 μg of 2; 12 μg of 1 and 100 μg of 2; 12 μg of 1 and 200 μg of 2; 12 μg of 1 and 300 μg of 2; 12 μg of 1 and 400 μg of 2; 12 μg of 1 and 500 μg of; 12 μg of 1 and 600 μg of 2; 12 μg of 1 and 700 μg of 2; 12 μg of 1′ and 800 μg of 2; 12 μg of 1′ and 900 μg of 2; 12 μg of 1 and 1000 μg of 2; 21.7 μg of 1 and 25 μg of 2; 21.7 μg of 1 and 50 μg of 2; 21.7 μg of 1 and 100 μg of 2; 21.7 μg of 1 and 200 μg of 2; 21.7 μg of 1 and 300 μg of 2; 21.7 μg of 1 and 400 μg of 2; 21.7 μg of 1′ and 500 μg of 2; 21.7 μg of 1 and 600 μg of 2; 21.7 μg of 1 and 700 μg of 2; 21.7 μg of 1 and 800 μg of 2; 21.7 μg of 1 and 900 μg of 2; 21.7 μg of 1 and 1000 μg of 2; 24.1 μg of 1 and 25 μg of 2; 24.1 μg of 1 and 50 μg of 2; 24.1 μg of 1′ and 100 μg of 2; 24.1 μg of 1 and 200 μg of 2; 24.1 μg of 1 and 300 μg of 2; 24.1 μg of 1 and 400 μg of 2; 24.1 μg of 1 and 500 μg of 2; 24.1 μg of 1 and 600 μg of 2; 24.1 μg of 1 and 700 μg of 2; 24.1 μg of 1 and 800 μg of 2; 24.1 μg of 1 and 900 μg of 2; 24.1 μg of 1 and 1000 μg of 2; 43.3 μg of 1 and 25 μg of 2; 43.3 μg of 1 and 50 μg of 2; 43.3 μg of 1 and 100 μg of 2; 43.3 μg of 1′ and 200 μg of 2; 43.3 μg of 1 and 300 μg of 2; 43.3 μg of 1 and 400 μg of 2; 43.3 μg of 1 and 500 μg of 2; 43.3 μg of 1 and 600 μg of 2; 43.3 μg of 1 and 700 μg of 2; 43.3 μg of 1′ and 800 μg of 2; 43.3 μg of 1′ and 900 μg of 2; 43.3 μg of 1 and 1000 μg of 2; 48.1 μg of 1 and 25 μg of 2; 48.1 μg of 1′ and 50 μg of 2; 48.1 μg of 1′ and 100 μg of 2; 48.1 μg of 1 and 200 μg of 2; 48.1 μg of 1′ and 300 μg of 2; 48.1 μg of 1′ and 400 μg of 2; 48.1 μg of 1′ and 500 μg of 2; 48.1 μg of 1′ and 600 μg of 2; 48.1 μg of 1 and 700 μg of 2; 48.1 μg of 1 and 800 μg of 2; 48.1 μg of 1 and 900 μg of 2 or 48.1 μg of 1 and 1000 μg of 2.

If the active substance combination in which 1 is tiotropium bromide monohydrate is used as the preferred combination of 1 and 2 according to the invention, the quantities of 1′ and 2 administered per single dose specified by way of example hereinbefore correspond to the following quantities of 1′ and 2 administered per single dose: 6.2 μg 1 and 25 μg 2; 6.2 μg 1 and 50 μg 2; 6.2 μg 1 and 100 μg 2; 6.2 μg 1 and 200 μg 2; 6.2 μg 1 and 300 μg 2; 6.2 μg 1 and 400 μg 2; 6.2 μg 1 and 500 μg 2; 6.2 μg 1 and 600 μg 2; 6.2 μg 1 and 700 μg 2; 6.2 μg 1 and 800 μg 2; 6.2 μg 1 and 900 μg 2; 6.2 μg 1 and 1000 μg 2; 12.5 μg 1 and 25 μg 2; 12.5 μg 1 and 50 μg 2; 12.5 μg 1 and 100 μg 2; 12.5 μg 1 and 200 μg 2; 12.5 μg 1 and 300 μg 2; 12.5 μg 1 and 400 μg 2; 12.5 μg 1 and 500 μg 2; 12.5 μg 1 and 600 μg 2; 12.5 μg 1 and 700 μg 2; 12.5 μg 1 and 800 μg 2; 12.5 μg 1 and 900 μg 2; 12.5 μg 1 and 1000 μg 2; 22.5 μg 1 and 25 μg 2; 22.5 μg 1 and 50 μg 2; 22.5 μg 1 and 100 μg 2; 22.5 μg 1 and 200 μg 2; 22.5 μg 1 and 300 μg 2; 22.5 μg 1 and 400 μg 2; 22.5 μg 1 and 500 μg 2; 22.5 μg 1 and 600 μg 2; 22.5 μg 1 and 700 μg 2; 22.5 μg 1 and 800 μg 2; 22.5 μg 1 and 900 μg 2; 22.5 μg 1 and 1000 μg 2; 25 μg 1 and 25 μg 2; 25 μg 1 and 50 μg 2; 25 μg 1 and 100 μg 2; 25 μg 1 and 200 μg 2; 25 μg 1 and 300 μg 2; 25 μg 1 and 400 μg 2; 25 μg 1 and 500 μg 2; 25 μg 1 and 600 μg 2; 25 μg 1 and 700 μg 2; 25 μg 1 and 800 μg 2; 25 μg 1 and 900 μg 2; 25 μg 1 and 1000 μg 2; 45 μg 1 and 25 μg 2; 45 μg 1 and 50 μg 2; 45 μg 1 and 100 μg 2; 45 μg 1 and 200 μg 2; 45 μg 1 and 300 μg 2; 45 μg 1 and 400 μg 2; 45 μg 1 and 500 μg 2; 45 μg 1 and 600 μg 2; 45 μg 1 and 700 μg 2; 45 μg 1 and 800 μg 2; 45 μg 1 and 900 μg 2; 45 μg 1 and 1000 μg 2; 50 μg 1 and 25 μg 2; 50 μg 1 and 50 μg 2; 50 μg 1 and 100 μg 2; 50 μg 1 and 200 μg 2; 50 μg 1 and 300 μg 2; 50 μg 1 and 400 μg 2; 50 μg 1 and 500 μg 2; 50 μg 1 and 600 μg 2; 50 μg 1 and 700 μg 2; 50 μg 1 and 800 μg 2; 50 μg 1 and 900 μg 2 or 50 μg 1 and 1000 μg 2.

The active substance combinations of 1 and 2 according to the invention are preferably administered by inhalation or by nasal application. For this purpose, ingredients 1 and 2 have to be made available in inhalable or nasal forms. Inhalable preparations include inhalable powders, propellant-containing metering aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A. Inhalable Powder Containing the Combinations of Active Substances 1 and 2 According to the Invention

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g., glucose or arabinose), disaccharides (e.g., lactose, saccharose, maltose), oligo- and polysaccharides (e.g., dextran), polyalcohols (e.g., sorbitol, mannitol, xylitol), salts (e.g., sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred. Within the scope of the inhalable powders according to the invention the excipients have a maximum average particle size of up to 250 μm, preferably between 10 μm and 150 μm, most preferably between 15 μm and 80 μm. It may sometimes seem appropriate to add finer excipient fractions with an average particle size of 1 μm to 9 μm to the excipients mentioned above. These finer excipients are also selected from the group of possible excipients listed hereinbefore. Finally, in order to prepare the inhalable powders according to the invention, micronised active substance 1 and 2, preferably with an average particle size of 0.5 μm to 10 μm, more preferably from 1 μm to 5 μm, is added to the excipient mixture. Processes for producing the inhalable powders according to the invention by grinding and micronizing and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630, or by other means as described in DE 36 25 685 A. Preferably, the inhalable powders according to the invention which contain physiologically acceptable excipient in addition to 1 and 2 are packed into capsules (to produce so-called inhalettes) which are used in inhalers as described, for example, in WO 94/28958.

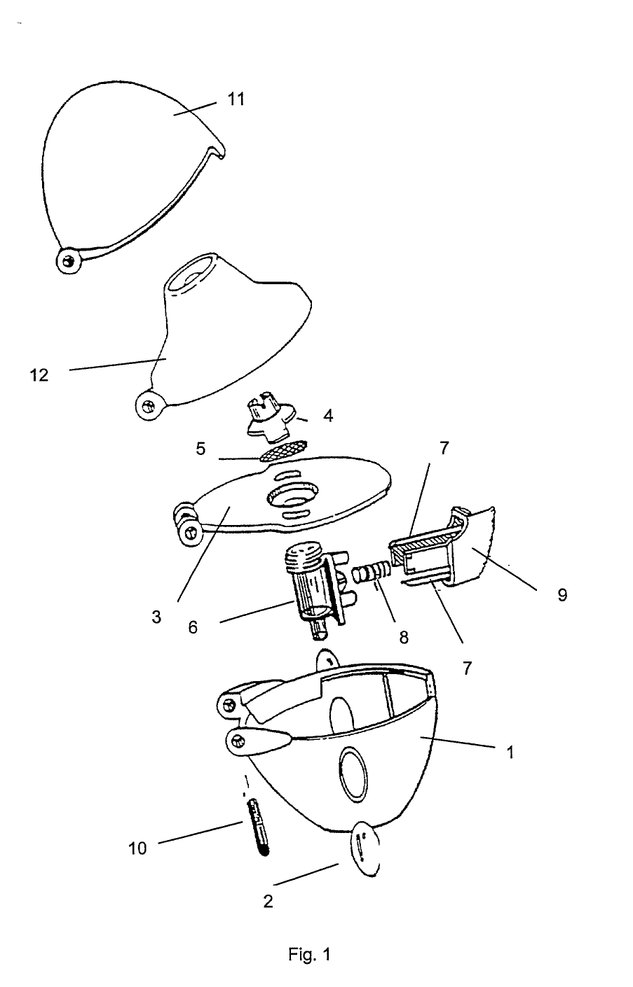

A particularly preferred inhaler for administering the pharmaceutical combination according to the invention in inhalettes is shown in FIG. 1.

This inhaler (Handihaler®) for inhaling powdered pharmaceutical compositions from capsules is characterized by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut.

If the inhalable powders according to the invention are packed into capsules (inhalers) for the preferred use described above, the quantities packed into each capsule should be 1 mg to 30 mg, preferably 3 mg to 20 mg, more particularly 5 mg to 10 mg of inhalable powder per capsule. These capsules contain, according to the invention, either together or separately, the doses of 1′ and 2 mentioned hereinbefore for each single dose.

B. Propellant Gas-Driven Inhalation Aerosols Containing the Combinations of Active Substances 1 and 2 According to the Invention

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG134a and TG227.

The propellant-driven inhalation aerosols according to the invention may also contain other ingredients such as co-solvents, stabilizers, surfactants, antioxidants, lubricants and pH adjusters. All these ingredients are known in the art.

The inhalation aerosols containing propellant gas according to the invention may contain up to 5 wt. % of active substance 1 and/or 2. Aerosols according to the invention contain, for example, 0.002 wt. % to 5 wt. %, 0.01 wt. % to 3 wt. %, 0.015 wt. % to 2 wt. %, 0.1 wt. % to 2 wt. %, 0.5 wt. % to 2 wt. %, or 0.5 wt. % to 1 wt. % of active substance 1 and/or 2.

If the active substances 1 and/or 2 are present in dispersed form, the particles of active substance preferably have an average particle size of up to 10 μm, preferably from 0.1 μm to 5 μm, more preferably from 1 μm to 5 μm.

The propellant-driven inhalation aerosols according to the invention mentioned above may be administered using inhalers known in the art (MDIs: metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C. Propellant-Free Inhalable Solutions or Suspensions Containing the Combinations of Active Substances 1 and 2 According to the Invention

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of suitable inorganic acids include hydrochloric acid, hydrobromic acid, nitric acid, sulfuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulfuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g., as flavorings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g., alcohols-particularly isopropyl alcohol, glycols-particularly propylene glycol, polyethylene glycol, polypropylene glycol, glycol ether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants and/or preservatives which guarantee or prolong the shelf life of the finished pharmaceutical formulation, flavorings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulizing a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 μL and 30 μL of active substance solution can be nebulized in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulizers (devices) described therein are known by the name Respimat®.

This nebulizer (Respimat®) can advantageously be used to produce the inhalable aerosols according to the invention containing the combination of active substances 1 and 2. Because of its cylindrical shape and handy size of less than 9 cm to 15 cm long and 2 cm to 4 cm wide, this device can be carried at all times by the patient. The nebulizer sprays a defined volume of pharmaceutical formulation using high pressures through small nozzles so as to produce inhalable aerosols.

The preferred atomizer essentially consists of an upper housing part, a pump housing, a nozzle, a locking mechanism, a spring housing, a spring and a storage container, characterized by

a pump housing which is secured in the upper housing part and which comprises at one end a nozzle body with the nozzle or nozzle arrangement;

a hollow plunger with valve body;

a power takeoff flange in which the hollow plunger is secured and which is located in the upper housing part;

a locking mechanism situated in the upper housing part;

a spring housing with the spring contained therein, which is rotatably mounted on the upper housing part by means of a rotary bearing; and

a lower housing part which is fitted onto the spring housing in the axial direction.

The hollow plunger with valve body corresponds to a device disclosed in WO 97/12687, which is incorporated herein by reference in its entirety. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 MPa to 60 MPa (about 50 bar to 600 bar), preferably 10 MPa to 60 MPa (about 100 bar to 600 bar) on the fluid, the measured amount of active substance solution, at its high pressure end at the moment when the spring is actuated. Volumes of 10 microliters to 50 microliters are preferred, while volumes of 10 microliters to 20 microliters are particularly preferred and a volume of 15 microliters per spray is most particularly preferred.

The valve body is preferably mounted at the end of the hollow plunger facing the valve body.

The nozzle in the nozzle body is preferably microstructured, i.e., produced by microtechnology. Microstructured valve bodies are disclosed, for example, in WO 94/07607; reference is hereby made to the contents of this specification, particularly FIG. 1 therein and the associated description. WO 94/07607 is incorporated herein by reference in its entirety.

The valve body consists for example of two sheets of glass and/or silicon firmly joined together, at least one of which has one or more microstructured channels which connect the nozzle inlet end to the nozzle outlet end. At the nozzle outlet end there is at least one round or non-round opening 2 microns to 10 microns deep and 5 microns to 15 microns wide, the depth preferably being 4.5 microns to 6.5 microns while the length is preferably 7 microns to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20° to 160° to one another, preferably 60° to 150°, most preferably 80° to 100°. The nozzle openings are preferably arranged at a spacing of 10 microns to 200 microns, more preferably at a spacing of 10 microns to 100 microns, most preferably 30 microns to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 bar to 300 bar, and is atomized into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g., a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomizer axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomizer; this causes the deformable ring to deform in the annual plane. Details of the construction of the locking mechanism are given in WO 97/20590.

The lower housing part is pushed axially over the spring housing and covers the mounting, the drive of the spindle and the storage container for the fluid.

When the atomizer is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g., 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomized may be pushed into the atomizer one after another and used in succession. The storage container contains the aqueous aerosol preparation according to the invention.

The atomizing process is initiated by pressing gently on the actuating button. As a result, the locking mechanism opens up the path for the power takeoff member. The biased spring pushes the plunger into the cylinder of the pump housing. The fluid leaves the nozzle of the atomizer in atomized form.

Further details of construction are disclosed in PCT Applications WO 97/12683 and WO 97/20590, to which reference is hereby made, and each of which is incorporated herein by reference in their entireties.

The components of the atomizer (nebulizer) are made of a material which is suitable for its purpose. The housing of the atomizer and, if its operation permits, other parts as well are preferably made of plastics, e.g., by injection moulding. For medicinal purposes, physiologically safe materials are used.

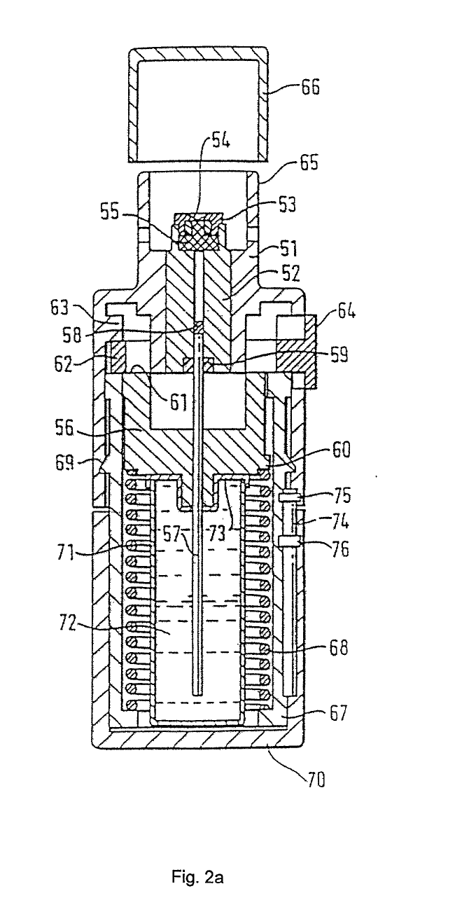

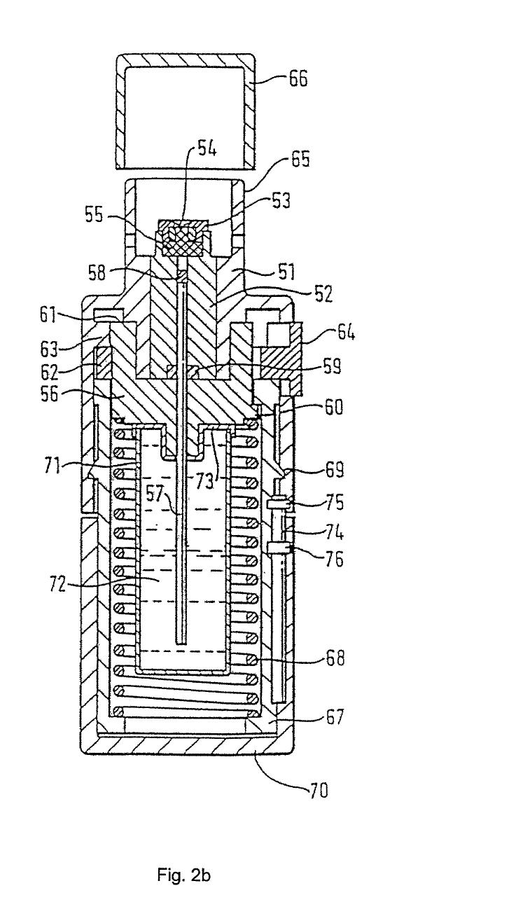

FIGS. 2a/b attached to this patent application, which are identical to FIGS. 6a/b of WO 97/12687, show the nebulizer (Respimat®) which can advantageously be used for inhaling the aqueous aerosol preparations according to the invention.

FIG. 2a shows a longitudinal section through the atomizer with the spring biased, while FIG. 2b shows a longitudinal section through the atomizer with the spring relaxed.

The upper housing part (51) contains the pump housing (52) on the end of which is mounted the holder (53) for the atomizer nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomized. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

The nebulizer described above is suitable for nebulizing the aerosol preparations according to the invention to produce an aerosol suitable for inhalation.

If the formulation according to the invention is nebulized using the method described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 mg and 30 mg of formulation, most preferably between 5 mg and 20 mg of formulation are delivered as a defined mass on each actuation.

However, the formulation according to the invention may also be nebulized by means of inhalers other than those described above, e.g., jet stream inhalers or other stationary nebulizers.

Accordingly, in a further aspect, the invention relates to pharmaceutical formulations in the form of propellant-free inhalable solutions or suspensions as described above combined with a device suitable for administering these formulations, preferably in conjunction with the Respimat®. Preferably, the invention relates to propellant-free inhalable solutions or suspensions characterized by the combination of active substances 1 and 2 according to the invention in conjunction with the device known by the name Respimat®. In addition, the present invention relates to the above-mentioned devices for inhalation, preferably the Respimat®, characterized in that they contain the propellant-free inhalable solutions or suspensions according to the invention as described hereinbefore.

The propellant-free inhalable solutions or suspensions according to the invention may take the form of concentrates or sterile inhalable solutions or suspensions ready for use, as well as the above-mentioned solutions and suspensions designed for use in a Respimat®. Formulations ready for use may be produced from the concentrates, for example, by the addition of isotonic saline solutions. Sterile formulations ready for use may be administered using energy-operated fixed or portable nebulizers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterized in that the device is an energy-operated free-standing or portable nebulizer which produces inhalable aerosols by means of ultrasound or compressed air by the Venturi principle or other methods.

The Examples which follow serve to illustrate the present invention in more detail without restricting the scope of the invention to the following embodiments by way of example.

Starting Materials

Tiotropium Bromide

The tiotropium bromide used in the following formulations examples may be obtained as described in European Patent Application 418 716 A1.

In order to prepare the inhalable powders according to the invention, crystalline tiotropium bromide monohydrate may also be used. This crystalline tiotropium bromide monohydrate may be obtained by the method described below.

15.0 kg of tiotropium bromide are placed in 25.7 kg of water in a suitable reaction vessel. The mixture is heated to 80° C. to 90° C. and stirred at constant temperature until a clear solution is formed. Activated charcoal (0.8 kg) moistened with water is suspended in 4.4 kg of water, this mixture is added to the solution containing the tiotropium bromide and the resulting mixture is rinsed with 4.3 kg of water. The mixture thus obtained is stirred for at least 15 minutes at 80° C. to 90° C. and then filtered through a heated filter into an apparatus preheated to an external temperature of 70° C. The filter is rinsed with 8.6 kg of water. The contents of the apparatus are cooled at 3° C.-5° C. for every 20 minutes to a temperature of 20° C.-25° C. The apparatus is cooled further to 10° C.-15° C. using cold water and crystallization is completed by stirring for at least another hour. The crystals are isolated using a suction filter dryer, the crystal slurry isolated is washed with 9 liters of cold water (10° C.-15° C.) and cold acetone (10° C.-15° C.). The crystals obtained are dried at 25° C. in a nitrogen current over a period of 2 hours. Yield: 13.4 kg of tiotropium bromide monohydrate (86% of theory).

The crystalline tiotropium bromide monohydrate thus obtained is micronised by known methods in order to prepare the active substance in the form of the average particle size corresponding to the specifications according to the invention.

### EXAMPLES OF FORMULATIONS

The examples of formulations for administration by inhalation as specified in A and B below may be prepared analogously to methods known in the art.

A. Inhalable Powders

| | |
|---|---|
| Ingredients | μg per capsule |

1. Inhalable Powder

| | |
|---|---|
| Tiotropium bromide | 21.7 |
| Epinastine hydrochloride | 200 |
| Lactose | 4778.3 |
| Total | 5000 |

2. Inhalable Powder

| | |
|---|---|
| Tiotropium bromide | 21.7 |
| Epinastine hydrochloride | 125 |
| Lactose | 4853.3 |
| Total | 5000 |

3. Inhalable Powder

| | |
|---|---|
| Tiotropium bromide × H₂O | 22.5 |
| Epinastine hydrochloride | 250 |
| Lactose | 4727.5 |
| Total | 5000 |

4. Inhalable Powder

| | |
|---|---|
| Tiotropium bromide | 21.7 |
| Epinastine hydrochloride | 250 |
| Lactose | 4728.3 |
| Total | 5000 |

5. Inhalable Powder

| | |
|---|---|
| Tiotropium bromide × H₂O | 22.5 |
| Epinastine hydrochloride | 495 |
| Lactose | 4482.5 |
| Total | 5000 |

6. Inhalable Powder

| | |
|---|---|
| Tiotropium bromide | 21.7 |
| Epinastine hydrochloride | 400 |
| Lactose | 4578.3 |
| Total | 5000 |

B. Propellant Gas-Containing Aerosols for Inhalation

| | |
|---|---|
| Ingredients | wt. % |

1. Suspension Aerosol

| | |
|---|---|
| Tiotropium bromide | 0.015 |
| Epinastine hydrochloride | 0.066 |
| Soya lecithin | 0.2 |
| TG 134a:TG227 (2:3) | to 100 |

2. Suspension Aerosol

| | |
|---|---|
| Tiotropium bromide | 0.029 |
| Epinastine hydrochloride | 0.33 |
| absolute ethanol | 0.5 |
| Isopropyl myristate | 0.1 |
| TG 227 | to 100 |

C. Forms for Nasal Administration

1. Solution

900 ml of purified water are placed in a suitable vessel and 285.7 mg of tiotropium monohydrate, 2000 mg of epinastine hydrochloride and 500 mg of disodium EDTA are dissolved therein with stirring. Then the pH of the solution is adjusted to 3 with 0.1 N hydrochloric acid and the solution is made up to a total volume of 1000 ml with purified water. The solution is transferred into a suitable pump for nasal use. With a spray volume of 70 μl per spray actuation, 20 μg of tiotropium bromide and 140 μg of epinastine hydrochloride are administered each time.

2. Powder

20 g of tiotropium bromide monohydrate and 140 g of epinastine hydrochloride with a particle size distribution for the two active substances containing about 90% of the active substance particles in the size range from 5 μm to 20 μlm are placed in a suitable mixer. 5.34 kg of lactose (200 M) are added to the two active substances and they are mixed together until a homogeneous mixture is obtained. Then 5.5 mg of this mixture are transferred into a nasal spray system. When administered nasally, 20 μg of tiotropium bromide and 140 μg of epinastine hydrochloride are delivered per spray.

We claim:

1. A pharmaceutical composition comprising:
(a) a tiotropium salt; and
(b) an antihistamine,
optionally together with a pharmaceutically acceptable excipient,
the tiotropium salt and the antihistamine optionally in the form of their enantiomers, mixtures of their enantiomers, their racemates, their solvates, or their hydrates.

2. The pharmaceutical composition according to claim 1, wherein the tiotropium salt is a salt with a counter-ion selected from chloride, bromide, iodide, p-toluene sulfonate, or methylsulfate.

3. The pharmaceutical composition of claim 2, wherein the counter-ion is bromide.

4. The pharmaceutical composition according to claim 1, wherein the antihistamine is selected from the group consisting of: epinastine, cetirizine, azelastine, fexofenadine, levocabastine, loratadine, mizolastine, ketotifen, emedastine, dimethindene, clemastine, bamipine, dexchlorpheniramine, pheniramine, doxylamine, chlorphenoxamine, dimenhydrinate, diphenhydramine, promethazine, ebastine, desloratadine, and meclozine.

5. The pharmaceutical composition according to claim 2, wherein the antihistamine is selected from the group consisting of: epinastine, cetirizine, azelastine, fexofenadine, levocabastine, loratadine, mizolastine, ketotifen, emedastine, dimethindene, clemastine, bamipine, dexchlorpheniramine, pheniramine, doxylamine, chlorphenoxamine, dimenhydrinate, diphenhydramine, promethazine, ebastine, desloratadine, and meclozine.

6. The pharmaceutical composition according to claim 3, wherein the antihistamine is selected from the group consisting of: epinastine, cetirizine, azelastine, fexofenadine, levocabastine, loratadine, mizolastine, ketotifen, emedastine, dimethindene, clemastine, bamipine, dexchlorpheniramine, pheniramine, doxylamine, chlorphenoxamine, dimenhydrinate, diphenhydramine, promethazine, ebastine, desloratadine, and meclozine.

7. The pharmaceutical composition according to claim 3, wherein the antihistamine is selected from the group consisting of: epinastine, cetirizine, azelastine, fexofenadine, levocabastine, loratadine, ebastine, desloratadine and mizolastine.

8. The pharmaceutical composition according to claim 1, wherein the weight ratios of the tiotropium salt to the antihistamine are in the range of from 1:300 to 50:1.

9. The pharmaceutical composition according to claim 8, wherein the weight ratios of the tiotropium salt to the antihistamine are in the range of from 1:250 to 40:1.

10. The pharmaceutical composition according to claim 1, wherein the pharmaceutical composition is an inhalable powder, a propellant-containing metering aerosol, or a propellant-free inhalable solution or suspension.

11. The pharmaceutical composition according to claim 1, wherein the pharmaceutical composition further comprises a suitable physiologically acceptable excipient selected from the group consisting of: monosaccharides, disaccharides, oligo- and polysaecharides, polyalcohols, and salts.

12. The pharmaceutical composition according to claim 2, wherein the pharmaceutical composition further comprises a suitable physiologically acceptable excipient selected from the group consisting of: monosaccharides, disaccharides, oligo- and polysaccharides, polyalcohols, and salts.

13. The pharmaceutical composition of claim 11, wherein the excipient has a maximum average particle size of up to 250 μm.

14. The pharmaceutical composition of claim 12, wherein the excipient has a maximum average particle size of up to 250 μm.

15. The pharmaceutical composition of claim 13, wherein the excipient has a maximum average particle size of between 10 μm and 150 μm.

16. The pharmaceutical composition of claim 14, wherein the excipient has a maximum average particle size of between 10 μm and 150 μm.

17. A capsule containing a pharmaceutical composition according to claim 1 in the form of an inhalable powder.

18. A capsule containing a pharmaceutical composition according to claim 2 in the form of an inhalable powder.

19. A capsule containing a pharmaceutical composition according to claim 3 in the form of an inhalable powder.

20. A capsule containing a pharmaceutical composition according to claim 4 in the form of an inhalable powder.

21. A capsule containing a pharmaceutical composition according to claim 5 in the form of an inhalable powder.

22. A capsule containing a pharmaceutical composition according to claim 6 in the form of an inhalable powder.

23. A capsule containing a pharmaceutical composition according to claim 7 in the form of an inhalable powder.

24. A capsule containing a pharmaceutical composition according to claim 8 in the form of an inhalable powder.

25. A capsule containing a pharmaceutical composition according to claim 9 in the form of an inhalable powder.

26. A capsule containing a pharmaceutical composition according to claim 10 in the form of an inhalable powder.

27. A capsule containing a pharmaceutical composition according to claim 11 in the form of an inhalable powder.

28. A capsule containing a pharmaceutical composition according to claim 12 in the form of an inhalable powder.

29. A capsule containing a pharmaceutical composition according to claim 13 in the form of an inhalable powder.

30. A capsule containing a pharmaceutical composition according to claim 14 in the form of an inhalable powder.

31. A capsule containing a pharmaceutical composition according to claim 15 in the form of an inhalable powder.

32. A capsule containing a pharmaceutical composition according to claim 16 in the form of an inhalable powder.

33. A pharmaceutical composition according to claim 1, where in the pharmaceutical composition is a propellant-containing inhalable aerosol and the tiotropium salt and the antihistamine are in dissolved or dispersed form.

34. The pharmaceutical composition according to claim 33, wherein the propellant-containing inhalable aerosol comprises a propellant gas selected from hydrocarbons and halohydrocarbons.

35. The pharmaceutical composition according to claim 33, wherein the propellant-containing inhalable aerosol comprises a propellant gas selected from the group consisting of: n-propane; n-butane; isobutane; and chlorinated and/or fluorinated derivatives of methane, ethane, propane, butane, cyclopropane, and cyclobutane.

36. The pharmaceutical composition according to claim 34, wherein the propellant gas is TG134a, TG227, or a mixture thereof.

37. The pharmaceutical composition according to claim 33, further comprising at least one of a cosolvent, stabilizer, surfactant, antioxidant, lubricant, or means for adjusting the pH of the composition.

38. The pharmaceutical composition according to claim 34, further comprising at least one of a cosolvent, stabilizer, surfactant, antioxidant, lubricant, or means for adjusting the pH of the composition.

39. The pharmaceutical composition according to claim 35, further comprising at least one of a cosolvent, stabilizer, surfactant, antioxidant, lubricant, or means for adjusting the pH of the composition.

40. The pharmaceutical composition according to claim 36, further comprising at least one of a cosolvent, stabilizer, surfactant, antioxidant, lubricant, or means for adjusting the pH of the composition.

41. The pharmaceutical composition according to claim 33, where in the amount of the tiotropium salt or the antihistamine is up to 5 wt. % of the pharmaceutical composition.

42. A pharmaceutical composition according to claim 1, wherein the pharmaceutical composition is propellant-free inhalable solution or suspension that further comprises a solvent selected from water, ethanol, or a mixture of water and ethanol.

43. The pharmaceutical composition according to claim 42, wherein the pH is between 2 and 7.

44. The pharmaceutical composition according to claim 43, wherein the pH is between 2 and 5.

45. The pharmaceutical composition according to claim 42, wherein the pH of the pharmaceutical composition is adjusted by means of one or more acids selected from the group consisting of: hydrochloric acid, hydrobromic acid, nitric acid, sulfuric acid, ascorbic acid, citric acid, malic acid, tartaric acid, maleic acid, succinic acid, fumaric acid, acetic acid, formic acid, and propionic acid.

46. The pharmaceutical composition according to claim 42, further comprising other co-solvents or excipients.

47. The pharmaceutical composition according to claim 45, further comprising other co-solvents or excipients.

48. The pharmaceutical composition according to claim 46, wherein the co-solvent is selected from the group consisting of alcohols, glycols, polyoxyethylene alcohols, and polyoxyethylene fatty acid esters.

49. The pharmaceutical composition according to claim 46, wherein the co-solvent is selected from the group consisting of: isopropyl alcohol, propylene glycol, polyethylene glycol, polypropylene glycol, glycol ether, and glycerol.

50. The pharmaceutical composition according to claim 46, wherein the excipient is selected from the group consisting of: surfactants, stabilizers, complexing agents, antioxidants, preservatives, flavorings, pharmacologically acceptable salts, and vitamins.

51. The pharmaceutical composition according to claim 50, wherein the excipient is selected from the group consisting of: edetic acid, a salt of edetic acid, ascorbic acid, vitamin A, vitamin E, tocopherols, cetyl pyridinium chloride, benzalkonium chloride, benzoic acid, and benzoate salts.

52. A pharmaceutical composition consisting essentially of:
(a) a tiotropium salt;
(b) an antihistamine;
(c) a solvent;
(d) benzalkonium chloride; and
(e) sodium edetate.

53. A pharmaceutical composition consisting essentially of:
(a) a tiotropium salt;
(b) an antihistamine;
(c) a solvent; and
(d) benzalkonium chloride.

54. A method of treating allergic or non-allergic rhinitis in a patient in need of such treatment, the method comprising administering to the patient a therapeutically effective amount of the pharmaceutical composition according to one of claims 1 to 12.

55. A kit comprising one or more unit dosage containers containing a pharmaceutical composition, each unit dosage container containing a pharmaceutical composition comprising:
(a) a tiotropium salt; and
(b) an antihistamine,
each optionally together with a pharmaceutically acceptable excipient,
the tiotropium salt and the antihistamine optionally in the form of their enantiomers, mixtures of their enantiomers, their racemates, their solvates, or their hydrates.

56. The kit according to claim 55, further comprising instructions with directions for using the kit.

57. A kit comprising:
(a) a first container containing a first pharmaceutical formulation comprising a tiotropium salt; and
(b) a second container containing a second pharmaceutical formulation comprising a comprising an antihistamine,
each container each optionally further containing a pharmaceutically acceptable excipient, the tiotropium salt and the antihistamine optionally in the form of their enantiomers, mixtures of their enantiomers, their racemates, their solvates, or their hydrates.

58. The kit according to claim 57, further comprising instructions with directions for using the kit.
